(19) United States
(12) Patent Application Publication
(10) Pub. No.: US 2023/0047417 A1
(43) Pub. Date: Feb. 16, 2023

(54) A SYSTEM FOR MONITORING AT LEAST ONE PROPERTY OF CONCRETE IN REAL TIME

(71) Applicant: Dmitry G. GORDI, Weston, FL (US)

(72) Inventor: Dmitry G. GORDI, Weston, FL (US)

(21) Appl. No.: 17/794,191

(22) PCT Filed: Jan. 22, 2021
(86) PCT No.: PCT/US2021/014711

§ 371 Date: Jul. 20, 2022

Related U.S. Application Data
(60) Provisional application No. 62/964,535, filed on Jan. 22, 2020.

(51) Int. Cl. G01N 3/06; H04W 4/38

(52) U.S. Cl. CPC G01N 3/06; H04W 4/38; G01N 2203/067

(57) ABSTRACT

The present invention is a system for measuring at least one property of concrete that comprises: a housing having an inner cavity, said housing being embeddable into concrete upon deployment of said system in the field; a controller having a form that fits inside said cavity, said controller being retrievable from said cavity after said system is deployed; a sensor detachably connected to the controller such that the sensor becomes embedded into concrete upon said deployment of said system; wherein said system is capable of measuring a property of concrete upon deployment. The present invention also provides a novel method of measuring the strength of concrete comprising attaching the system as described above to a supporting element of a construction structure; immersing said housing and said sensor into concrete whereby the sensor is completely embedded inside the concrete; and measuring data related to the strength of concrete using the sensor and then transmitting the data wirelessly to a remote server.

### CROSS-REFERENCE TO RELATED APPLICATIONS

This application claims priority under 35 U.S.C. 119(e) to U.S Provisional Application Ser. No. 62/964,535 filed on Jan. 22, 2020, which is incorporated by reference, in its entirety.

### FIELD

The present invention relates to a system for measuring and reporting at least one property of concrete and monitoring the process of concrete solidification and life-time structural stability. The system comprises a sensor, a controller containing electronic components and a wireless data transmitter. The present invention also relates to a method of embedding or immersing the system inside concrete in such a manner that it can relay an unobstructed signal regarding the state of concrete to a remote server.

### BACKGROUND

It is important for contractors and engineers to evaluate the strength of concrete hardening in real-time and to have an appreciation for the strength of newly constructed concrete structures. This ensures that further construction can proceed as early as possible and yet is a safe manner. Additionally, it is important for contractors and engineers to be able to evaluate the integrity of the completed structures throughout the entire period of their use from the completion of construction to the demolition. Under normal circumstances, concrete curing usually takes 28 days. However, due to a variety of environmental conditions including ambient temperature of the air, soil, and precipitation and the specific composition of the concrete used, concrete curing may take between 15-90 days. Thus, solutions that provide reliable and accurate state of concrete before, during, and after the concrete curing process are desired.

The existing methods of concrete strength monitoring are expensive, inaccurate and inconvenient. These methods usually include collecting the concrete samples at the construction site, curing of the samples in the lab to simulate real-time curing at the construction site, and measuring the strength of the concrete sample by compressing cylinders over time. Many such tests require the use of a laboratory. Recently, non-destructive testing methods that use sensors that measure the strength of concrete at the construction sites have been developed. Most common of these methods is the temperature testing, which utilizes a thermocouple. The method is also called the maturity method reflecting the fact that a user following the temperature profile of the solidifying concrete can generally determine when it will solidify. However, maturity method is burdensome on users because it requires recalibration for every new concrete mixture.

The existing temperature testing equipment for concrete includes such examples as Giatec's Smart Rock™, Lumicon's LumiNode™, Converge's Converge Signal™, and Hilti's concrete sensors. None of these sensors have a replaceable controller and battery. Some of these devices use Internet of Things (hereinafter referred to as “IoT”) solutions to transmit the signal from the buried sensor within the concrete to an outside Bluetooth or wireless device that is capable to receiving the data from which the strength of concrete can be calculated. The embedded devices are designed to transmit their measurements from within the concrete, throughout the concrete curing process, to an outside device.

A problem that these devices face is that concrete is a poor conductor of a wireless signal thus necessitating a close proximity of a user with the receiver to the embedded device. The existing devices embedded in concrete cannot effectively transmit wireless signals accurately. That makes it difficult to utilize the wireless technology in the field of concrete strength and quality control, as receiver needs to be brought in close proximity to the device immersed in concrete to obtain data.

US2017/0284996 entitled “Embedded Wireless Monitoring Sensors” discloses a system for continuous temperature monitoring of the poured concrete with wireless interface comprising non-removable boxes containing temperature sensors, a wireless transceiver and a battery. The publication further discloses that the resulting data may be transmitted to a portable electronic device such as cellular telephone or a portable computer or a fixed electronic device that relies for power of electrical utilities.

The above system along with some other largely analogous systems in this field suffer from several other major disadvantages, including inability to remove and reuse the most expensive parts of the system which are electronic components and further inability to change a dead battery upon which the system relies for its functioning. This undermines the continuity of the system's operation and renders it unable to monitor the conditions within the concrete structure over the life of the structure. Moreover, the existing systems do not effectively overcome the problem of concrete being a poor transmitter of the wireless signal. Therefore, while the disclosures of such systems mention wireless transmission, in practice they are largely limited or even entirely non-enabled because any wireless transmission from the sensors quickly deteriorates upon increase in concrete thickness. U.S. Pat. Nos. 10,775,332; 10,768,130; 10,571,418; 10,324,078; 9,804,111; 9,638,652; 6,690,182.

A more accurate method for measuring the solidification progress of concrete and its strength and integrity over the life of the structure is ultrasonic testing. The ultrasonic testing utilizes high-frequency sound waves that are transmitted throughout the material being tested in order to conduct a thorough inspection. An ultrasonic wave is a mechanical vibration or pressure wave similar to audible sound, but with a much higher vibration frequency usually above about 20 kHz. Ultrasonic inspection can be used to detect surface flaws, such as cracks, seams, and internal flaws such as voids or inclusions of foreign material. It's also used to measure wall thickness in tubes and diameters of bars. Depending on the test requirements, these waves can be highly directional and focused on a small spot or thin line, or limited to a very short duration. An ultrasonic pulse velocity (UPV) test is an in-situ, nondestructive test to check the quality of concrete and natural rocks. In this test, the strength and quality of concrete or rock is assessed by measuring the velocity of an ultrasonic pulse passing through a concrete structure or natural rock formation. This testing method is outlined in ASTM D597.

This test is conducted by passing a pulse of ultrasonic through concrete to be tested and measuring the time taken by pulse to get through the structure. Ultrasonic testing equipment includes a pulse generation circuit, consisting of electronic circuit for generating pulses and a transducer for transforming electronic pulse into mechanical pulse having an oscillation frequency in the range of greater than 20 kHz, and a pulse reception circuit that receives the signal.

The major disadvantage of the existing ultrasonic testing equipment is its high cost. An industrial grade ultrasonic sensor designed to estimate the state of a substance or a material with real time online access currently costs about 7,000 euros in Europe and a comparable amount in the United States. Handheld ultrasonic equipment for concrete is from one to three thousand euros each, depending on functionality and brand. It must be operated by a highly professional employee, which significantly adds to the cost. Additionally, the existing handheld devices are only suitable for surface measurements as they must be held against the surface. They are incapable of being embedded or immersed into concrete. Finally, each pulse of a handheld device is sent by the user who must press the trigger button.

The existing ultrasonic testing equipment for concrete includes such examples as Humboldt's H-2984.XX, HC-6320.XX, HC-6450, HC-6451, HC-6485, H-2880, HC-6440, HC-6390. None of the Humbold's testing equipment can be embedded or immersed in concrete and thus is only suitable for measuring the exposed surfaces which limits its accuracy and requires physical presence of the user on site who manually performs the measurements.

The present invention solves the problems discussed above by providing a system and method for determining at least one property of concrete, including the strength of concrete. The system of the present invention is capable of performing from one measurement to tens of thousands of measurements per minute accumulating that data thus greatly increasing accuracy of the final summary data that is sent to the server. The system of the present invention does not require the presence of the user. The system of the present invention costs orders of magnitude less. The system of the present invention can be accurately and properly positioned in the structure by construction workers as a matter of their routine workflow while installing the wall formwork and can stay in place for a lifetime monitoring of the completed structure subject only to battery exchange. And if the lifetime monitoring is not required, the valuable controller may be removed and reused in other construction locations or construction projects.

The present system can use a variety of sensors based on different principles, but in a particularly preferred embodiment uses ultrasonic sensors. The system also contains an IoT component that allows sending an unobstructed signal from within the concrete to a remote server/user.

The present invention is a system for measuring at least one property of concrete that comprises: a housing having an inner cavity, said housing being embeddable into concrete upon deployment of said system in the field; a controller having a form that fits inside said cavity, said controller being retrievable from said cavity after said system is deployed; a sensor located outside said housing and detachably connected to the controller such that the sensor becomes embedded into concrete upon said deployment of said system; wherein said system is capable of measuring a property of concrete upon deployment. The present invention also provides a novel method of measuring the strength of concrete comprising attaching the system as described above to a supporting element of a construction structure; immersing said housing and said sensor into concrete whereby the sensor is completely embedded inside the concrete; and measuring data related to the strength of concrete using the sensor and then transmitting the data wirelessly to a remote server.

### SUMMARY

The present invention describes a system for measuring at least one property of concrete, said system comprising: a housing having an inner cavity, said housing being embeddable into concrete upon deployment of said system in the field; a controller having form that fits inside said cavity, said controller being retrievable from said cavity after said system is deployed; a sensor detachably connected to the controller such that becomes embedded into concrete upon said deployment of said system; wherein said system is capable of measuring a property of concrete upon deployment.

The system as described above, where the controller and the sensor are placed within the same housing.

The system as described above, where the sensor is outside the housing.

The system as described above, where said controller is reusable.

The system as described above, wherein the sensor is the one selected from a group consisting of ultrasonic sensor, temperature sensor, pressure sensor, humidity sensor, electrical resistance sensor, light sensor, acceleration sensor, vibration sensor, pH sensor, ion content sensor, chloride content sensor, microphone sensor, acoustic sensor, gas sensor, corrosion sensor, and hardness sensor.

The present invention describes a method of wirelessly measuring the strength of concrete during construction by deploying the system as described above, said method comprising: attaching said system to a supporting element of a construction structure; immersing said housing and said sensor into concrete whereby the sensor is completely embedded inside the concrete; and measuring data related to the strength of concrete using the sensor.

The method as described above, wherein the controller is attached to the sensor by means of the connecting wire.

The method as described above, further comprising transmitting of data by the embedded sensor to the controller through the connecting wire; the controller transmitting the data to a remote server.

The method as described above, wherein the controller transmitting the data is through LoRa WAN, SigFOx, Wi-Fi or any other wireless short range or long range connection.

The method as described above, further comprising: receiving of the data by the remote server; processing and computing the strength of concrete maturity using the received data.

The method as described above, wherein the supporting element of a construction structure is a rebar.

The method as described above, wherein the supporting element is a tie rod.

### BRIEF DESCRIPTION OF THE DRAWINGS

FIG. 1 illustrates an embodiment of the invention comprising the controller in a housing and the sensor at some distance from the sensor.

FIG. 2 illustrates an embodiment of the invention comprising the controller and the sensor in a shared housing forming a compact unit.

FIG. 3 is a 3D illustration of the embodiment of FIG. 2.

FIG. 4a illustrates the method of attaching the controller along with the outer case to the rebars before pouring wet concrete; 46b illustrates how the components of tie rods that correspond to the outer plugs of the invention are visible (black holes) on the outer surface of the concrete slab.

### DETAILED DESCRIPTION OF THE INVENTION

The invention is defined with reference to the appended claims. With respect to the claims, the glossary that follows provides the relevant definitions.

Strength of concrete according to the invention refers to the state or status of concrete at any point from loading the concrete by a constructor to his truck, during transportation, at the site of construction, before mixing with water and gravel, after mixing, before pouring into the formwork, during the process of curing, hardening, after completely forming/curing, over the years during its routine wear and tear and other circumstance where rapid deterioration is expected. Field according to the invention is any construction or construction related area where monitoring the properties of concrete is required.

“Spacer tubes for tie rods,” as used in this specification, are straight plastic tubes of a circular cross-section which are routinely used in the construction industry to encase and protect the tie rods.

A “tie rod,” as used in this specification, is a metal rod with two clamps or screws on each end that is designed to keep the wall framework in vertical position and to provide the appropriate spacing between the sheets of the framework that will correspond to the thickness of the wall. Sometimes rebar is used as the metal rod. Said tubes are routinely used in modern construction. Typically, a construction site would have standard spacing tubes for tie rods readily available. During construction of walls, said tubes are used to encase the tie rods and prevent the tie rods from being entrapped in solidified concrete. The tubes allow the tie rods to be removed from them when the concrete reaches the solid state. The tubes remain encased in the solidified concrete and are not removed. Construction workers install the tie rods and the spacing tubes for tie rods as a matter of course during the construction, because the tie rods help to maintain the framework in place. To assure that the system retains its ability to recycle the controller and the battery, it is important that the outer plug is always exposed and observable to a user on the outside wall after the work is finished and concrete is in the process of solidification or is completely solidified.

A sensor according to the invention measures one or more properties of the concrete and is able to transmit the measured data to the controller. The sensor according to the invention is selected from a group consisting of ultrasonic sensor, temperature sensor, pressure sensor, humidity sensor, electrical resistance sensor, light sensor, acceleration sensor, vibration sensor, pH sensor, ion content sensor, chloride content sensor, microphone sensor, acoustic sensor, gas sensor, corrosion sensor, and hardness sensor. A sensor according to the invention is any sensor available in the market that measures a property of concrete. In a preferred embodiment, the sensor according to the invention is constructed and specially adapted for the system of the present invention. In a preferred embodiment of the invention, the sensor is an ultrasonic sensor comprising one or more transducers.

The ultrasonic sensor according to the invention measures the strength of concrete around the area of its transducer. In a preferred embodiment, the ultrasonic sensor according to the present invention measures the strength of concrete between the areas of its two transducers. The sensor is then able to transmit the measured data to the controller either through a wired connection or wirelessly. The ultrasonic sensor according to the invention comprises of an emitter and a receiver. The sensor according to the invention is connected to the controller and placed in such a manner that its transducers are immersed into the wet concrete. Upon measuring, the sensor has the ability to transmit the data to the controller in a continuous manner. The sensor according to the invention continues to measure the property of concrete even after the concrete is completely cured.

In an embodiment of the invention, the sensor is connected to the controller by means of a connecting wire. In an alternative embodiment of the invention, the sensor according to the invention is connected to the controller wirelessly, wherein the wireless connection is any industry accepted wireless protocol or system. The sensor measures a property of concrete and conveys the measured data to the controller.

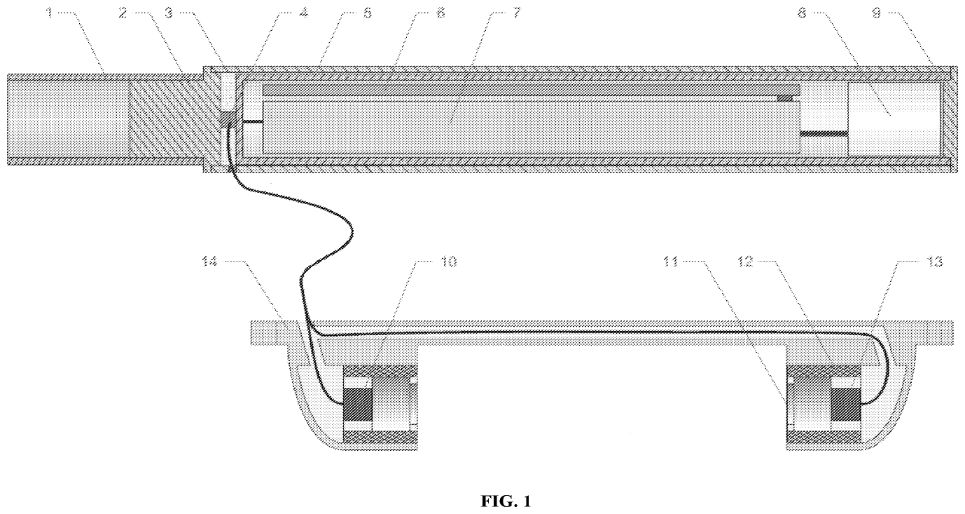

In a preferred embodiment of the invention, as shown in FIG. 1, the sensor of the invention comprises components 10-14. In this embodiment, the sensor is at some distance away from the controller, connected through a wire. This embodiment allows the measurement of the concrete at much deeper level than where the controller is placed. Depending on the requirement of a particular project, a user may prefer this embodiment although it is not as compact. The sensor in this embodiment comprises a transducer 11, a DC-DC converter 10, a vibration insulator 12, signal amplifier 13 and transducers casing 14. In this embodiment, the sensor measures the strength of concrete that is placed between the transducers 11 on either side. Additionally, the vibration insulator 12 protects the transducer from mechanical stress caused during the construction.

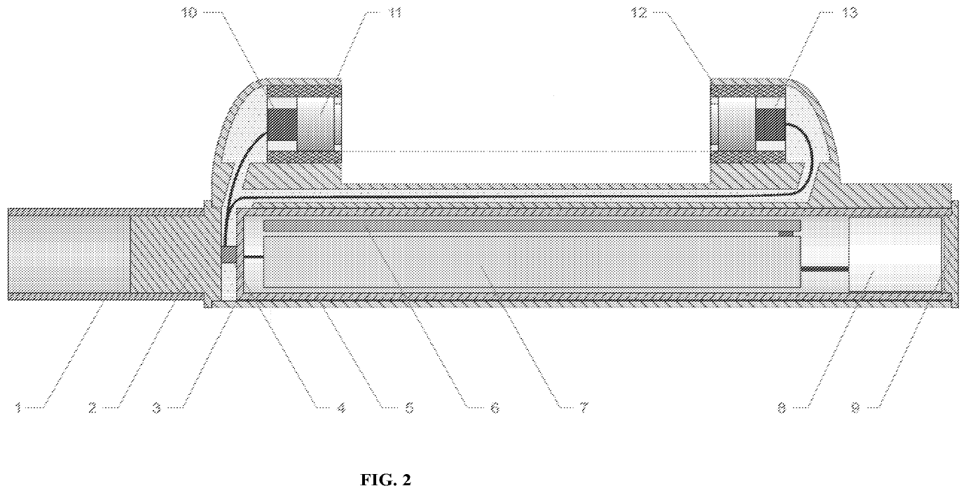

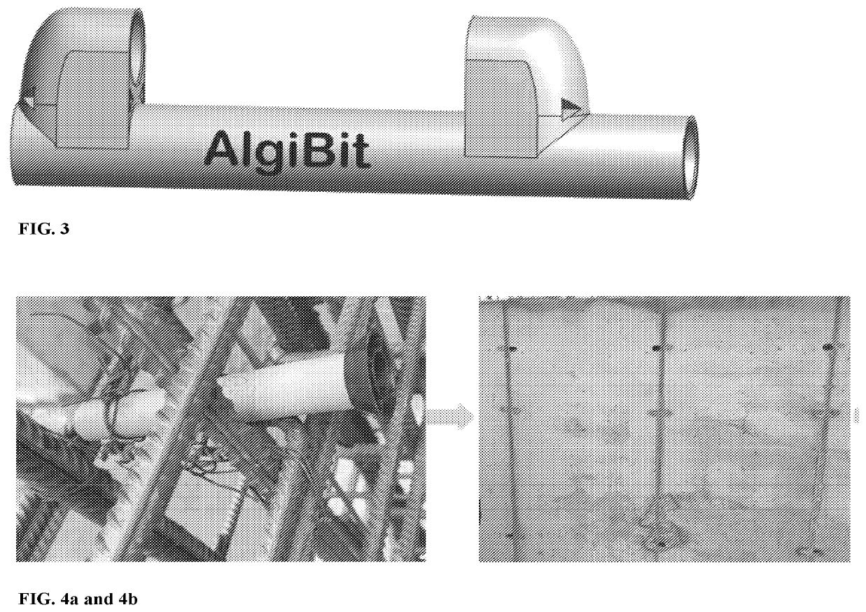

In an alternate embodiment of the invention, as shown in FIG. 2, the sensor of the invention is present in a single housing along with the controller. This embodiment provides a more compact model that removes the presence of any outside wires prone to damage during the construction process. Depending on the requirement of a particular project, a user may prefer this embodiment over the embodiment of FIG. 1.

The controller according to the invention receives the measured data from the sensor. The controller according to the invention is reusable. The controller according to the invention is not embedded directly into the concrete. The controller according to the invention is placed inside a housing that protects the controller from physically coming into contact with the concrete. The controller comprises a controller case that holds its internal components as a single unit. The controller case of the controller may be of any shape so long as it can be easily placed and removed from the housing. In an embodiment of the invention the controller case is elongated in shape. In a preferred embodiment of the invention, the internal components of the controller comprise a processor, a transmission device, an antenna and other relevant electronic components. In a preferred embodiment, the controller is connected to the sensor by the means of a wired connection. The controller receives the data from the sensor by means of the wired connection. The controller is detachable from the sensor by unplugging the wire connecting said controller to the sensor. In an alternate embodiment, the controller is wirelessly connected to the sensor, wherein the controller receives data from the sensor wirelessly using any standard wireless protocol.

The controller according to the invention is a combination of an oscilloscope and IoT network data transfer module. In an embodiment of the invention, the controller comprises the following elements inside a controller case: an antenna, a plug to connect to the sensor, battery, and related electronics that enable receiving and transmitting the information from the sensor. The controller receives the measured data from the ultrasonic sensor embedded in the concrete. The controller has the ability to store the measured data, process the data, or transmit the raw unprocessed data to an outside cloud server or an equivalent computing system that can receive said data. The controller is placed in such a manner that there is no concrete blocking its signal to the server from at least one end of the controller. The controller is placed in a housing that is attached to a rebar or other stable structures at a construction site before pouring the wet concrete. After the concrete is cured and the formwork is removed, the controller can still transmit data from the sensor for prolonged periods of time. The housing may be opened to replace the battery power of the controller if required. Where prolonged supervision of concrete structures is not required, the controller may be unplugged from the sensor and removed from the housing to be reused. The controller according to the invention transmits the sensor data through LoRa WAN, SigFOx, Wi-Fi or any other wireless short range or long-range connection to a remote server for data processing and transmission to the end-user device.

In a preferred embodiment of the invention, as illustrated in FIG. 1, the controller comprises of antenna 6, controller electronics 7, plug to the connecting wire 4, batteries 8 arranged so as to form a single unit inside a controller case 5. The controller case 5 according to this embodiment is further enclosed by an outer case 3, which outer case may be elongated in length by connecting it with a spacer tube 1 by means of an inner plug 2. As an example, a spacer tube could be a PVC spacer tube that is ordinarily used through formwork for housing tie rods, and also known as tie rod sleeve. The purpose of using the longer spacer tube is to assure that the system can be securely affixed to a rebar by, e.g., wire and also additionally be held in place by the wall formwork and therefore firmly maintain its position and not be swept away by the flow of the heavy and dense concrete.

The housing according to the invention protects the controller and its electronic components from the mechanical and chemical degradation in the concrete. The housing is an elongated structure with an inner cavity that consists of an outer surface and an inner surface, a proximate end and a distal end. In a preferred embodiment, the length of the housing may be adapted to the length of the concrete block being constructed. As discussed above, the length may be extended to correspond with the thickness of the concrete wall or the length of the concrete block by using spacer tubes that may be attached at one end of the system. In the same embodiment, at least one end of the housing may be opened or closed by a protective means such as protective caps. In this embodiment, the housing is tied to a stable structural element of the framework such as a rebar with the controller placed inside the housing, wherein the controller in turn is connected to the sensor of the invention.

Typical housing consists of rugged materials similar to a pipe sleeve for tie rod protection, typically used in concrete formworks. The housing material of the invention offers sufficient protection to the controller with the wall thickness of said housing being the same as of the pipe sleeves or greater. The materials from which said housing could be made include but not limited to metals (from which the contacts are isolated by insulating rings etc.), ceramics (e.g. alumina, zirconia, etc.), composites (e.g. fiber reinforced polymer, ceramic matrix composites, concrete, glass-reinforced plastic, etc.) and plastics (e.g. short-fiber thermoplastics, long-fiber thermoplastics, thermosetting plastics, filled plastics, synthetic rubber, elastomer, etc.), poly-vinyl chloride (PVC), polypropylene (PP), polyamide (Nylon®), polyurethane (PU), polyethylene, high density polyethylene (HDPE), ethyl vinyl acetate, polytetrafluoroethylene (PTFE), polyvinylidene fluoride (PVDF), polyketones (PEEK, PEK, and PEKK), polyoxybenzylmethylenglycolanhydride (Bakelite®), and various rubber or silicon materials. It is understood that in addition to the housing, various other components of the system can be made of these materials. For example, the plugs can be made from either elastic materials like rubber or silicon or from plastic. Typical cross-section of housing includes circular, oval, square etc. and can also have smooth even outside or inside surfaces or, alternatively, can have corrugated or uneven surfaces. One skilled in the art would understand that having corrugated surfaces increases rigidity of the housing and therefore its protective ability while allowing for thinner walls.

In a preferred embodiment of the invention, the housing comprises an outer case 3 preinstalled around the controller. The controller in this embodiment contains an inner plug 2 that may be used to attach a spacer tube of variable length in order to extend the length of the entire controller to correspond to the length of the concrete slab. The controller in this embodiment also contains an outer plug 9, which plug may be opened by a user to replace a battery or simply to retrieve the controller after completion of the project.

In a preferred embodiment of the invention, the housing facilitates unobstructed transmission of signal from the controller to the remote server or other outside computing device. For example, because the length of the case corresponds to the thickness of the concrete wall or a concrete structure, upon removal of the formwork, the controller via its antenna, is able to transmit a signal unobstructed by concrete at least from the end of the concrete slab with the outer plug 9. Additionally, the housing is also situated in a manner that in cases where prolonged monitoring of concrete is required, a user has the option of opening the outer plug to replace the battery. Additionally, the housing also allows a user to open the outer plug and retrieve the controller after conclusion of the monitoring for the purpose of reusing the controller.

Deployment according to the invention is attaching the system as described above in any of its described embodiments, to a structural element of the framework such that it can start measuring the strength of concrete. Deployment is the manner in which the system of the present invention is positioned before it is immersed under the concrete. In a preferred embodiment of the invention, the system is deployed when the housing is attached to a rebar at the site of construction with the controller placed inside the housing, and wherein the controller is connected to the sensor. The system of the present invention is attached to the existing framework (typically comprising rebars in the reinforced concrete structures) in such a way that it ensures that a cone shaped cap at the end of the housing (or the outer plugs in FIG. 1) are located right at the outer edge of the concrete wall, allowing for wireless connection signal to travel freely, without being obstructed by concrete. See FIGS. 4a and 4b

A supporting element of the invention is any structural element in the construction work or construction related work where measurement of concrete properties is desired. For example, supporting elements of the invention include but not limited to rebars, tie rods.

Remote server according to the invention is a cloud-based server or a cloud service. In an embodiment of the invention, the measured data of concrete from the sensor is received by a controller and wirelessly transmitted to a remote server. The remote server can be used for the collection, analysis, storage, processing and retrieval of the data. In one embodiment of the present invention, a remote server employs at least one additional software program. A software program according to the embodiment is any program that can process the data received from the sensor, correlate said data with pre-determined reference standards, and determine current state and strength of concrete. Alternatively, the server may use artificial intelligence for data analysis. The collected data is presented to a user via website or specialized software that may be accessible on a mobile phone, a tablet, a laptop or a desktop computer.

In an embodiment of the invention, the system for measuring at least one property of concrete comprises: a housing having an inner cavity, said housing being embeddable into concrete upon deployment of said system in the field; a controller having a form that fits inside said cavity, said controller being retrievable from said cavity after said system is deployed; a sensor located outside said housing, and detachably connected to the controller such that the sensor becomes embedded into concrete upon said deployment of said system; wherein said system is capable of measuring the property of concrete upon deployment.

FIG. 1 describes an embodiment of the system according to the invention where the sensor is located at some distance away from the housing with the controller. In FIG. 1, the controller is the upper portion of the figure with components labeled 1-9; whereas the lower portion depicts the sensor with its components labeled 11-14. The controller and sensor are connected by means of a connecting wire 4 which transmits the concrete measurement data from the sensor to the controller. The figure shows the controller with its components: antenna 6, controller electronics 7, and batteries 8, arranged to form a single unit within the controller case 5. The antenna 6 aids in transmission of data by way of a wireless signal to a remote server. The antenna 6 aids in transmission of data by way of a wireless signal to a remote cloud server. The battery 8 serves an energy supplier to the system. The controller case 5 is the reusable part of the system. The controller case 5 is in turn enclosed in an outer case 3, where one end of the outer case has an outer plug 9 that may be opened if required, in order to retrieve the controller 7 or replace the battery 8. These outer plugs are visible in the concrete wall after removal of the formwork. For example FIG. 4b shows a concrete wall after removal of formwork, where black pipe cones (which correspond to the outer plugs here) are visible within the wall with their surface unobstructed by concrete. The other end of the controller has an inner plug, which inner plug is used to add a spacer tube 1 if required to extend the length of the entire controller from one end of the concrete slab to the other. In other words, the inner plug 2 serves as an adapter and sealant and separates the outer case 3 that contains the controller 7 from the detachable spacer 1. The controller is located within a controller case 5. The sensor is encased in sensor's casing 10 which contains the AC/DC converter 11, the transducer 12, the vibration insulator 13, and the signal amplifier 14.

FIG. 2 illustrates an embodiment of the present invention wherein the controller and the sensor are placed within the same housing, the numbers 1-14 correspond to the same elements as described for FIG. 1.

The use of the spacing tubes for tie rods, cut to match the thickness of the wall under construction, allows the system of the present invention to remain in place and assures that its ends are exposed and observable at all times. The tube is assured to stay firmly in place by being tied to a rebar in several places and additionally by the compression forces of the wall framework upon the ends of the tube.

In an embodiment of the invention, the method of wirelessly measuring the strength of concrete during construction by deploying the system described above comprises attaching said system to a supporting element of a construction structure; immersing said housing and said sensor into concrete whereby the sensor is completely embedded inside the concrete; and measuring data related to the strength of concrete using the sensor.

The deployment of the system is achieved by embedding or immersion of the system into unsolidified concrete, or, alternatively, affixing the system to the elements of the supporting structure (e.g. rebar) before the concrete is poured. In one embodiment of the present invention the housing should be the same length as the width of the constructed concrete wall or another structure where the device is expected to be deployed, as this approach guarantees that the two ends of the housing are going to be visible on each side of the wall and accessible to a user. The installation is pictured in FIGS. 4a and 4b.

When the measurements are no longer needed, the user may remove the protective cap, similar to the way cone shaped caps are removed from tie rod sleeves, unplug the connection to the sensor embedded in concrete, and retrieve the controller case with electronics. The case with electronics becomes reusable.

It is a notable advantage of the system over the prior art that it remains in place at specific, preselected and known to the user non-random locations. Such specific locations allow the system to generate uniquely useful data about the strength of the concrete under investigation. A yet further advantage is that the system remains in place and neither the valuable controller or the sensor are displaced by concrete pouring and lost at an undetermined location. A further substantial advantage of the system and its method of use lies in its user-friendly application. The system leverages the experience of a construction worker who is very familiar with installation of spacing tubes for tie rods. It is this familiarity that ensures consistency and reliability of installation and therefore enhanced reliability of the obtained data.

While the invention has been described above with reference to specific embodiments thereof, it is apparent that many changes, modifications, and variations can be made without departing from the inventive concept disclosed herein, and such description is not intended as limitations on the scope thereof. Accordingly, it is intended to embrace all such changes, modifications, and variations that fall within the spirit and broad scope of the appended claims.

What is claimed is:

1. A system for measuring at least one property of concrete, said system comprising:
a. a housing having an inner cavity, said housing being embeddable into concrete upon deployment of said system in the field;
b. a controller having form that fits inside said cavity, said controller being retrievable from said cavity after said system is deployed;
c. a sensor detachably connected to the controller such that said sensor becomes embedded into concrete upon said deployment of said system;
wherein said system is capable of measuring the property of concrete upon deployment.

2. The system of claim 1, wherein the sensor is located outside said housing.

3. The system of claim 1, where the controller and the sensor are placed within the same housing.

4. The system of claim 1, where said controller is reusable.

5. The system of claim 1, wherein the sensor is any sensor that measures at least one property of concrete.

6. The system of claim 1, wherein the sensor is selected from a group consisting of ultrasonic sensor, temperature sensor, pressure sensor, humidity sensor, electrical resistance sensor, light sensor, acceleration sensor, vibration sensor, pH sensor, ion content sensor, chloride content sensor, microphone sensor, acoustic sensor, gas sensor, corrosion sensor, and hardness sensor.

7. The system of claim 1, wherein the sensor is an ultrasonic sensor.

8. The system of claim 1, wherein the sensor is a temperature sensor.

9. The system of claim 1, wherein the sensor is a humidity sensor.

10. The system of claim 1, wherein the sensor is a pressure sensor.

11. The system of claim 1, wherein the sensor is a chloride content sensor.

12. The system of claim 1, wherein the sensor is a corrosion sensor.

13. The system of claim 1, wherein the sensor is a hardness sensor.

14. A method of wirelessly measuring the strength of concrete during construction by deploying the system of claim 1, said method comprising:
a. attaching said system to a supporting element of a construction structure;
b. immersing said housing and said sensor into concrete whereby the sensor is embedded inside the concrete; and
c. measuring data related to the strength of concrete using the sensor.

15. The method of wirelessly measuring the strength of concrete during construction by deploying the system of claim 2, said method comprising:
a. attaching said system to a supporting element of a construction structure;
b. immersing said housing and said sensor into concrete whereby at least a portion of the sensor is embedded into the concrete; and
c. measuring data related to the strength of concrete using the sensor.

16. The method of claim 14, wherein the controller is attached to the sensor by means of the connecting wire.

17. The method of claim 14, further comprising:
a. transmitting data by the embedded sensor to the controller through the connecting wire;
b. the controller transmitting the data to a remote server.

18. The method of claim 14, wherein the controller transmits the data through LoRa WAN, SigFOx, Wi-Fi or any other wireless short range or long range connection.

19. The method of claim 14, further comprising:
a. receiving of the data by the remote server;
b. processing and computing the strength of concrete maturity using the received data.

20. The method of claim 14, wherein the supporting element of a construction structure is a rebar.

21. The method of claim 14, wherein the supporting element of a construction structure is a tie rod.
